(19) United States
(12) Patent Application Publication
(10) Pub. No.: US 2014/0046436 A1
(43) Pub. Date: Feb. 13, 2014

(54) IMPLANTABLE PROSTHETIC VALVES AND METHODS

(71) Applicant: THE REGENTS OF THE UNIVERSITY OF CALIFORNIA, Oakland, CA (US)

(72) Inventor: Arash Kheradvar, Irvine, CA (US)

(73) Assignee: THE REGENTS OF THE UNIVERSITY OF CALIFORNIA, Oakland, CA (US)

(21) Appl. No.: 13/951,315

(22) Filed: Jul. 25, 2013

Related U.S. Application Data
(60) Provisional application No. 61/676,425, filed on Jul. 27, 2012.

(51) Int. Cl. A61F 2/24

(52) U.S. Cl. CPC A61F 2/2412
USPC 623/2.14; 623/2.18

(57) ABSTRACT

A prosthetic valve including an annulus, a pair of leaflets, and a pair of support elements is described. The annulus has a generally saddle-type shape and is connected to the support elements. The pair of leaflets extends from the annulus and are separated from each other by the support elements. In use, the valve is open when the support elements are angled or separated outward, and sealed or closed when the support elements are angled or moved inward.

### CROSS-REFERENCE TO RELATED APPLICATION

This filing claims the benefit of and priority to U.S. Patent Application Ser. No. 61/676,425, filed Jul. 27, 2012, which is incorporated by reference herein in its entirety for all purposes.

### GRANTS

The subject studies were supported by American Heart Association Grant No. 10BGIA4170011.

### BACKGROUND

Human heart valves under the conditions of normal physiological functions are organs that open under changes in pressure gradient inside the cardiac chambers. Four valves in the heart serve to direct the flow of blood through all chambers in a forward direction. When disease conditions affect the structure or the materials of the native valve, the valve itself will decay, degenerate, or disrupt, and requires repair or replacement to restore proper function necessary for the continuation of life.

U.S. Pat. No. 7,331,991 to Kheradvar, et al., discloses an implantable prosthetic valve that is transformable from a first helical pre-implantation configuration to a second valvular functional configuration and associated methods of delivery.

US Patent Publication No. 2009/0164003 to Kheradvar, alone, describes another type of prosthetic valve, excerpts of which are reproduced herein in connection with FIGS. 1A-2B. This valve was further publicly described at the 2010 Scientific Session of the American Heart Association and the 6th Biennial Meeting of The Society for Heart Valve Disease held in Barcelona on Jun. 25-28, 2011, as illustrated in FIGS. 3A-3C.

Though superficially similar to the latter example, the prosthetic valve described below is clearly distinguished in terms of relevant configuration and operation. As such, the subject prosthetic valve provides another option in prosthetic design and construction for addressing the ongoing need for improved implantable prosthetic valves that can satisfactorily replace native valves.

### SUMMARY

A prosthetic valve is provided that includes an annulus, a pair of leaflets, and a pair of support elements. The annulus has a generally saddle-type shape optionally formed by a movable pair of first portions separated from each other by a movable pair of second portions. The pair of leaflets extends from the annulus and each leaflet is separated from each other by the pair of support elements. The first portions of the annulus and the second portions of the annulus are configured to move back and forth.

The movement may be from being generally concave to being generally convex such that any movement of the first portions of the annulus occurs at generally the same time as any movement of the second portions of the annulus. In this example, the pair of leaflets are configured to define an opening when the first portions of the annulus are each generally convex and the second portions are separated to a maximum extent.

If the annulus states are not fully concave and convex (as further described herein) in use, then they will be relatively more concave and convex in comparison to one another in the different valve configuration states (i.e., open and closed). Thus, if in the open state the valve annulus is flat or near-flat, it will have transitioned from a closed state in which the valve is concave in use. Likewise, if in the closed state the value annulus is flat or near flat, it will have transitioned from an open state in which the valve is convex in use.

Embodiments of the subject hardware, the methods of its operation, and the methods of implanting such a valve are described herein. The method of implantation including delivering the subject prosthetic valve to an implant site in a patient and deploying it at the implant site.

Other systems, devices, methods, features, and advantages of the subject matter described herein will be or will become apparent to one with skill in the art upon examination of the following figures and detailed description. It is intended that all such additional systems, devices, methods, features, and advantages be included within this description, be within the scope of the subject matter described herein, and be protected by the accompanying claims. In no way should the features of the example embodiments be construed as limiting the appended claims, absent express recitation of those features in the claims.

### BRIEF DESCRIPTION OF THE DRAWINGS

The figures provided herein are diagrammatic and not necessarily drawn to scale, with some components and features exaggerated and/or abstracted for clarity. Variations from the embodiments pictured are contemplated. Accordingly, depiction of aspects and elements in the figures are not intended to limit the scope of the claims, except when such intent is explicitly stated as such.

FIGS. 1A and 1B illustrate an embodiment of the prosthetic valve from US Publication No. 2009/0164003 in its closed configuration from side and perspective views, respectively. FIGS. 2A and 2B FIG. 2 illustrates the same embodiment in its open configuration from side and perspective views, respectively. FIGS. 3A-3C are perspective view snapshots of a Finite Element Analysis (FEA) animation of a model of the same embodiment.

FIGS. 4A and 4B are perspective and side assembly views of an example embodiment of the valve, respectively. FIG. 5 is a top view of a completed assembly of an example embodiment of the valve in its open configuration and closed configuration.

FIGS. 6A and 6B show an example embodiment of the valve in open and closed configurations during use in an anatomical model.

FIGS. 7A and 7B are side views (orthogonal to each another) of another example embodiment of a valve. FIGS. 8A and 8B are side views (orthogonal to each another) of another example embodiment of a valve.

FIG. 9A is a graph of angular motion of support elements or members for the embodiments of FIGS. 7A, 7B, 8A, and 8B. FIG. 9B is a graph of saddle displacement for the embodiments of FIGS. 7A, 7B, 8A, and 8B.

### DETAILED DESCRIPTION

Before the present disclosure is described in detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

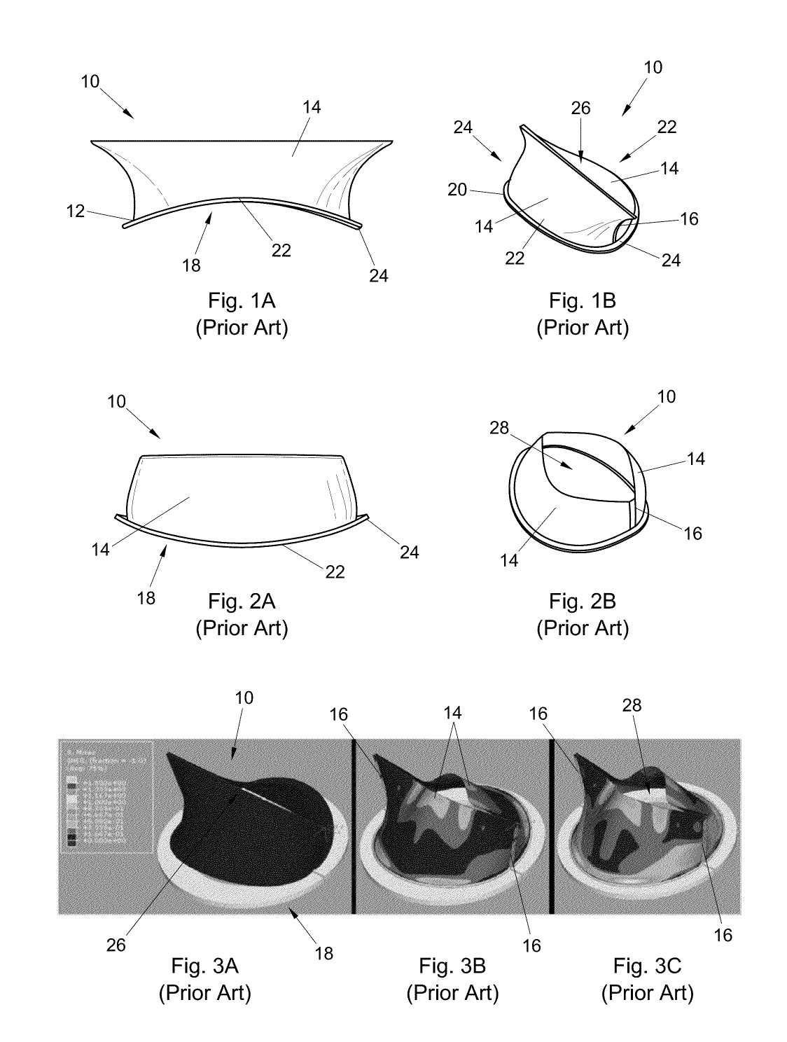

Before providing such discussion, however, the subject matter in and related to US Patent Publication No. 2009/0164003 noted above is described. FIGS. 1A and 1B illustrate this prosthetic valve 10 in its closed configuration from side and perspective views, respectively. FIGS. 2A and 2B illustrate the same valve in its open configuration from side and perspective views, respectively.

In these figures, valve 10 includes a body 12. The body 12 can include an annulus 18 having a generally saddle-shaped periphery 20 wherein the term “saddle-shaped” generally refers to a geometry in which periphery 20 includes one or more ridges that connect two higher elevations.

Periphery 20 includes pairs of first periphery portions 22 and second periphery portions 24. The periphery portions 22, 24 are movable with each of the first periphery portions 22 being separated from one another by each of the second periphery portions 24, and vice versa. The first periphery portions 22 are designed to move at generally the same time as the second periphery portions 24, and vice versa. The periphery portions 22, 24 can be integral to form periphery 20 or can be separately formed from one or more suitable materials and joined together using any suitable method as would be understood in the art.

Valve 10 also includes a pair of leaflets 14 and a pair of support elements 16. The leaflets and support elements extend from the superior surface of the body 12. Each leaflet is separated from another by a support element 16, and vice versa. Support elements 16 can be of any suitable shape, such as a rod shape as illustrated. They may assume a curved shape as shown in FIGS. 1A-B and 2A-B or be more linear as shown in FIGS. 3A-B.

Leaflets 14 can be joined to the support elements 16 by any suitable method as known by (or would be apparent to) those in the art such as by stitching, adhesives, or the like. Similarly, leaflets 14 and support elements 16 can each be joined to body 12 by any suitable method as known by (or would be apparent to) those in the art.

Body 12, annulus 18, and periphery 20 are configured so as to enable movement at approximately the same time between first portions of the annulus and second portions of the annulus so that when each of the first annulus portions 22 are in a concave position, each of the second portions 24 are in a convex position, and vice versa.

When the prosthetic valve 12 is in its closed configuration, as depicted in FIGS. 1A and 1B, aspects of each second periphery portion 24 that are attached to each support element 16 are concave, and the support elements 16 are curved appropriately outward to keep the distal ends of the leaflets 14 tight to (in contact with) each other to define a seal 26, obstructing the flow from distal to proximal end of the valve 12 and maintaining the unidirectional function of the valve. Meanwhile, the first periphery portions 22 are each generally convex establishing an overall generally saddle-shape for the periphery 20.

When the valve 12 is in its open configuration, as depicted in FIGS. 2A and 2B, the aspects of each second periphery portion 24 that are attached to each support element 16 are deflected to a convex shape, and the support elements 16 are curved inward maximizing the distance between the distal ends of the leaflets 14 to maintain the utmost size of the opening 28 defined by the leaflets 14 to allow unidirectional flow. At generally the same time that the second periphery portions 24 move into a convex shape, the first periphery portions 22 each move into a generally concave shape establishing an overall generally saddle-shape geometry for the periphery 20.

In this regard, valve 10 is designed so that the saddle-shaped annulus can transform its shape due to pressure change. When the first periphery portions of the annulus moves from being generally concave to being generally convex, the second periphery portions of the annulus move from being generally convex to being generally concave, and vice versa.

FIGS. 3A-3C illustrate the effect of such action for the valve embodiment in FIGS. 1A-B and 2A-B. FIG. 3A includes a legend (common to each of FIGS. 3A-C) for Von Mises stress distribution over the studied valve components. As progressively shown in FIGS. 3B and 3C, inward deflection of the support elements 16 drive an outward “puckering” and opening of the valve leaflets 14, given their structural configuration, or adaptation, and connection to each other. Stresses are greatest and the opening 28 size largest in FIG. 3C.

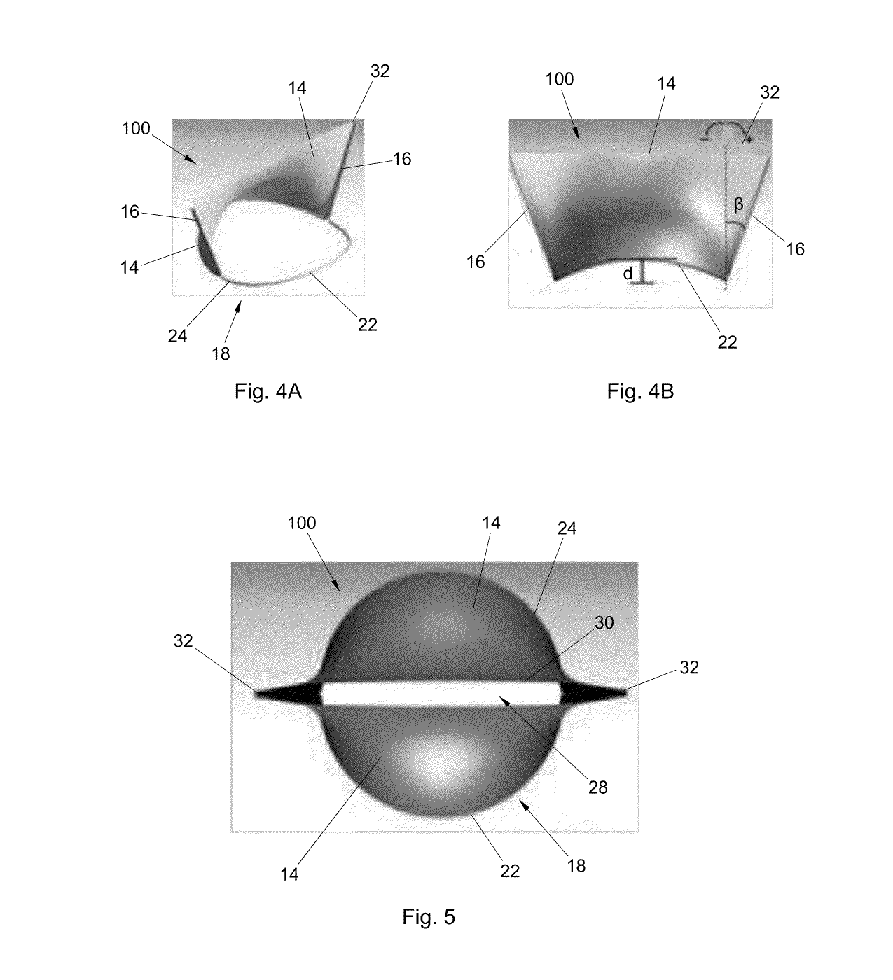

FIGS. 4A and 4B are perspective and side assembly views of the subject valve 100, respectively. Similar components to those described above are numbered identically. Structural differences between valve 10 and valve 100 are most apparent in FIG. 5, which illustrates the completed valve assembly 100. Here, support elements 16 and connected leaflets cooperate to define a slot 30 for an opening 28 when the first periphery portions 22 are convex. Note the offset distance “d” in FIG. 4B that will be discussed later in this regard.

Notably, support elements 16 are separated to their maximum extent (to which position they may be naturally biased by the material annuls and support element frame configuration—such as may be achieved by heat-setting Nitinol). These may pivot +/− about an angle β as shown in FIG. 4B. The angle range may vary depending on valve configuration and intended use as illustrated by the Examples below. In any case, terminal ends 32 of the support elements are most separated at the greatest positive angle and least at the greatest negative angle, as illustrated. Also, the degree of curvature of saddle convexity (shown convex-up in the direction of the leaflets and concave-down away from the leaflets) when open may vary in light of application.

In use, implanted at the mitral valve position, blood passes through slot 30 when the valve is located in the Mitral valve position. In systole, the annulus and support elements change configuration at the urging of fluid pressure and anatomical change to the valve seat within the heart to which the prosthetic valve is attached in order to bring support elements closer and allow leaflets to coapt and close the valve to flow.

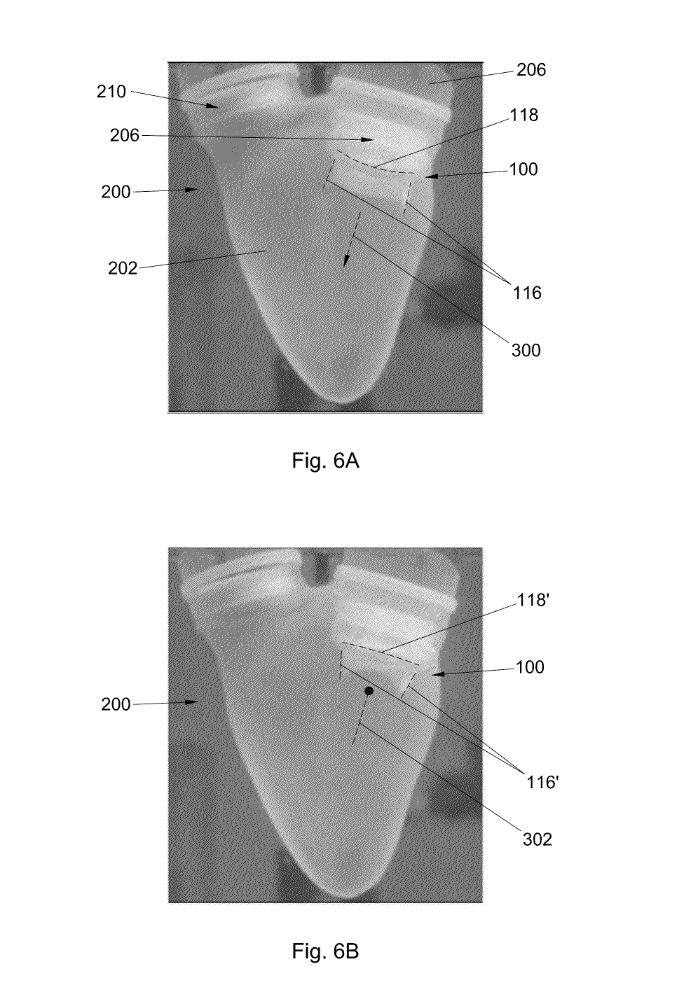

These states and configurations of valve 100 are shown in FIGS. 6A and 6B. The open valve state is depicted in FIG. 6A; the closed state of the valve is depicted in FIG. 6B. When implanted in a heart—the condition that model 200 simulates for in vitro hemodynamic study—pressure changes therein drive valve annulus shape and overall valve configuration as indicated from the model testing.

As for the model, it includes a thin-walled ventricle 202, shaped according to the systolic state, and is made from transparent silicone rubber. The ventricular sac was suspended over a plexiglas atria 204 to be free-floating inside a rigid, water-filled, container made from plexiglas (not shown) and connected to a hydraulic pump system (Superpump system; Vivitro Systems Inc., Victoria, BC, Canada—not shown). The pump was controlled by a customized interface that regulated the motion of the pump's piston according to predefined waveforms that were automatically adjusted based on the position, velocity and pressure feedback received by a power amplifier (SPA3891Z; Vivitro Systems Inc.—not shown). Pumping waveforms induced the desired trans-mitral suction and trans-aortic forward flow in the silicone ventricle during the cardiac cycle. Distilled water was used as the circulating fluid.

In each of FIGS. 6A and 6B, the approximate shape of valve support elements 16 viewed from the side is indicated by overlay 116/116′ and the orientation of valve annulus 18 by overlay 118/118′. The different configurations of the valve (i.e., open and closed, respectively) are thereby highlighted.

Flow through the valve in the model's ventricular diastole condition as enabled by valve opening is indicated by the arrow 300 in FIG. 6A. The stoppage of flow by valve seal and closure in the model's ventricular systole condition is indicated by the terminated flow line 302.

With changes in the valve annulus concavity as shown, changes in support element configuration work the leaflets. The valve leaflets are held open in the FIG. 6A configuration (just as shown in FIG. 5). They are slackened so the material can shift inward closing the valve in complementary fashion as in FIG. 6B. In doing so, the leaflet material may fold or wrinkle somewhat as they coapt.

Yet, valve closure is achieved without substantial prolapse and/or regurgitation during use. The following examples of valve construction and dimension were investigated and are provided herein to ensure such performance.

### EXAMPLES

Exemplary valves 100L (Long) and 100S (Short) were constructed and operated in the model shown in FIGS. 6A and 6B. These embodiments were designed to have 11 mm and 25 mm leaflet lengths to exemplify two extreme cases based on human mitral valve data.

To reproduce the cardiac cycle, a waveform to create a systolic ratio (SR) of 40% was used to imitate the ventricular flow conditions. The SR is the fraction of time in a cardiac cycle during which the left ventricle is in systolic phase. The frequency of cycles was set to different values ranging from 1 Hz to 1.67 Hz (60-100 beats per min) to reproduce an operational range for cardiac function. Each experiment was set to run for 10s (10-16 cardiac cycles) to ensure the consistency and reproducibility of the results. The atrial contraction phase was not reproduced by this waveform.

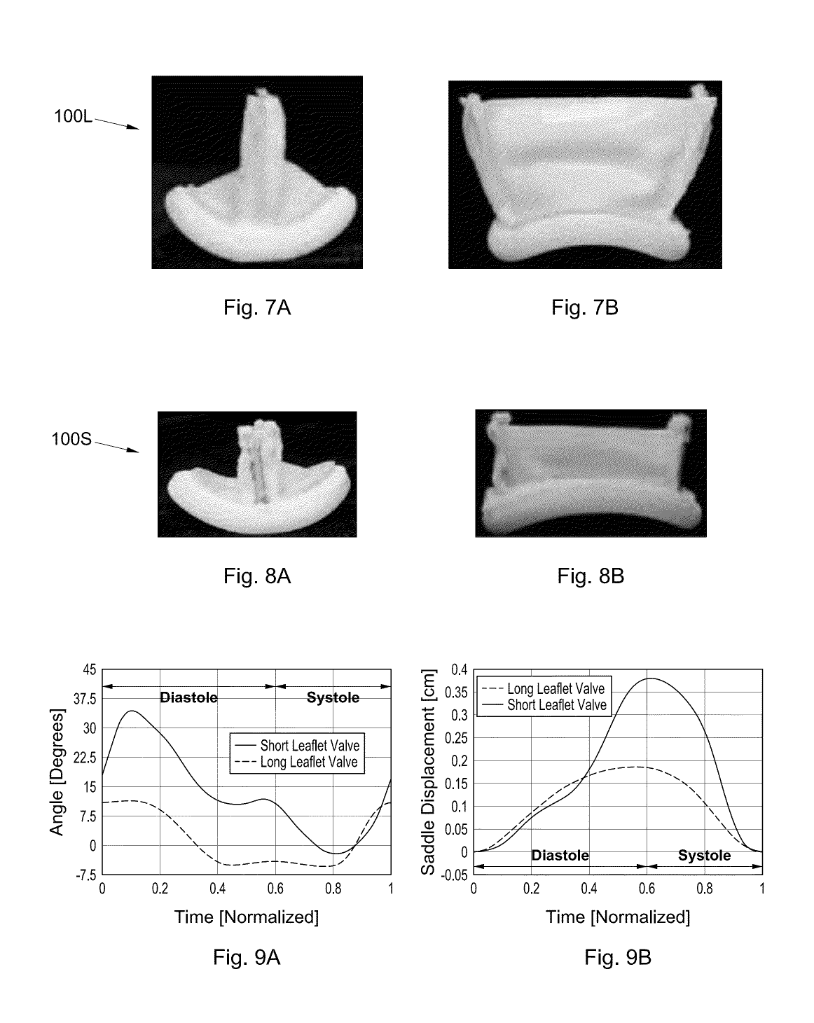

Data was produced using hemodynamic flow simulator 200 (shown in FIGS. 6A/6B), in which bileaflet mitral prototypes 100L and 100S (shown in FIGS. 7A-B and 8A-B, respectively) and a control trileaflet valve (SJM Biocor™; St. Jude Medical, Inc., Minn., USA—not shown) were placed at the mitral position 206, and a 23 mm Sorin Biomedica Carbocast mechanical heart valve (CarboMedics, Austin, Tex., USA) was placed at the aortic position 208.

Dynamic motion of the natural mitral valve is due to the elastic composition of its fibrous annulus. To imitate the motion of the mitral annulus, superelastic Nitinol wires were used with 8% strain recovery, shaped into a saddle-shape annulus with two support member prongs for attachment and holding the leaflets. The annulus was sutured to the proximal ends of the leaflets, which were made from 0.5 mm (average thickness) bovine pericardial tissue. The leaflets and the saddle-shaped annulus were also sutured to each other by along Nitinoal support elements extended from the annulus alongside the leaflets. The valve characteristics, such as annulus height (comparing 11 mm to 25 mm height), and resulting annulus curvature (starting with an apex distance “d” of 3.25 mm per FIG. 4B) and the changing support element angle were observed by constraining the Nitinol wire to a specialized housing unit designed for an adult heart with an annulus diameter of 25 mm.

Valve annulus motion was captured during the cardiac cycle through high-speed imaging (1030 fps; 1280×1024). The tip of the saddle's movement and the angle of motion of the support elements were measured based on tracking the objects using the image-processing toolbox of MATLAB (MathWorks, Inc.). These results are shown in FIGS. 9A and 9B.

Per above, when the valve was opening during diastole (FIG. 6A), the saddle annulus demonstrated a convex shape at the sides, while the supporting prongs were angled or curved outwards, keeping the leaflets far from each other and maintaining the utmost orifice size (e.g., as in FIG. 5). When the valve was closed during systole, the saddle annulus was deflected back to a less convex or even flat shape or concave shape (as depicted by overlay 118′), while the supporting prongs became angled or curved inwards so as to minimize the orifice and prevent regurgitation.

Regarding the angle of motion (β) of the support elements, when the valve was in the fully relaxed mode (i.e., at least partially open), the angle β was +20.20° and +11.25° for the short-leaflet and long leaflet valves, respectively. During systole, the distal end of the elements began to move towards the center of the valve, and this resulted in smaller values of β. When the end of the support element(s) passed an imaginary line at the intersection of the annulus saddle and the prongs, the angle β turned negative according to the sign convention. Based on these experiments, the angle of motion changed in the range of −3 to 35° and −5 to 11.25° in the short-leaflet and long-leaflet valves, respectively. During systole, the saddle tips of both valves underwent axial displacement, changing from a convex to a flatter or flattened shape. The maximum displacements of the saddle apex for the short-leaflet and long-leaflet valves were 3.85 mm and 3.65 mm, respectively.

These tests and data clearly demonstrate the valve operation as intended for a large anatomical size range in an in vitro setting. Although this testing provides a basis for the assertion that the mitral leaflet length may be varied from 11 to 25 mm, based on left ventricular size, the optimal length of the leaflets may be better defined based on data acquired from animal studies and future human clinical trials. As noted in “The Effects of Dynamic Saddle Annulus and Leaflet Length on Transmitral Flow Pattern and Leaflet Stress of a Bileaflet Bioprosthetic Mitral Valve”, A. Kheradvar, A. Falahatpisheh, J. Heart Valve Dis. Vol. 21. No. 2, March 2012 (incorporated herein by reference in its entirety for all purposes), stresses observed from modeling may indicate that shortening the leaflets beyond 11 mm may lead to an incomplete leaflet cooptation, regurgitation, or even valve prolapse, whereas lengthening the leaflets beyond 25 mm may result in interference with the chordae tendineae and left ventricular outflow tract obstruction.

Variations

These and other modifications and variations to the present disclosure can be practiced by those of ordinary skill in the art, without departing from the spirit and scope of the present disclosure, which is more particularly set forth in the appended claims. In addition, it should be understood that aspects of the various embodiments can be interchanged in whole or in part. Furthermore, those of ordinary skill in the art will appreciate that the foregoing description is by way of example only, and is not intended to limit the disclosure.

That being said, the material or tissue for the leaflets can comprise a compliant membrane of a single sheet or a compound manifold. In certain embodiments, the pericardial tissue can be selected from the group consisting of bovine, equine, porcine, ovine, human tissue, or combinations thereof. The leaflets can be made of any suitable synthetic material, engineered biological tissue, biological valvular leaflet tissue, pericardial tissue, or crosslinked pericardial tissue. In certain embodiments, the pericardial tissue may be procured from bovine, equine, porcine, ovine, human, or other animals. In certain embodiments, the crosslinked pericardial tissue is crosslinked with a crosslinking agent selected from the group consisting of formaldehyde, glutaraldehyde, dialdehyde starch, glyceraldehydes, cyanamide, diimides, diisocyanates, dimethyl adipimidate, carbodiimide, epoxy compound, and mixture thereof.

The annulus components and the support elements may comprise Nitinol employed for its shape memory effect or for superelasticty at human body temperature. Each of the elements described herein can have its own size, height, shape, or construction material according to the need of the prosthetic valve or implantation sites. The prosthetic valve should be formed so as to have the required rigidity, stability, biocompatibility, and substantially seamless characteristics (such as leak-proof property) to support a functional valve.

Further optional constructional details, those regarding suitable coatings and delivery options that may be utilized can be drawn from the '003 publication incorporated by reference, above. Likewise, the subject implants may be delivered or otherwise constructed as described in U.S. Provisional Patent Application No. 61/835,083, filed Jun. 14, 2013, which is also incorporated by reference herein in its entirety for all purposes.

The subject methods may include each of the physician activities associated with implant positioning, re-positioning, retrieval and/or release. The valve has a functional valvular configuration when it is deployed inside the heart, a blood vessel, a lymphatic vessel, or other body channel. Notably, the implantation site may be at any of a venous system, an esophagus, a stomach, a ureter, a urethral, a biliary passage, or an intestine. The procedure for delivering the valve includes a percutaneous manner, an endoscopic manner, a laparoscopic manner, a trans-apical manner, and the like. For instance, the valve can be implanted surgically or delivered with minimally-invasive means such as through a major vessel such as femoral artery, carotid artery, jugular vein, subclavian vein, femoral vein, or any other suitable blood vessel. In certain heart valve replacement applications, the valve can be delivered and deployed in an open-chest operation, optionally combined with other surgical procedures. Regarding these methods, as well as methods of manufacture and use that also form embodiments hereof, these may be carried out in any order of the events which is logically possible, as well as any recited order of events.

Furthermore, it is contemplated that any optional feature of the inventive variations described may be set forth and claimed independently or in combination with any one or more of the features described herein. And although the invention has been described in reference to several examples, various changes may be made to the invention described and equivalents (whether recited herein or not included for the sake of some brevity) may be substituted without departing from the true spirit and scope hereof.

Reference to a singular item includes the possibility that there are a plurality of the same items present. More specifically, as used herein and in the appended claims, the singular forms “a,” “an,” “said,” and “the” include plural referents unless specifically stated otherwise. In other words, use of the articles allow for “at least one” of the subject item in the description above as well as the claims below. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Without the use of such exclusive terminology, the term “comprising” in the claims shall allow for the inclusion of any additional element—irrespective of whether a given number of elements are enumerated in the claim, or the addition of a feature could be regarded as transforming the nature of an element set forth in the claims. Except as specifically defined herein, all technical and scientific terms used herein are to be given as broad a commonly understood meaning as possible while maintaining claim validity.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges is also encompassed within the disclosure, subject to any specifically excluded limit in the stated range. Where the stated range includes one or both of the limits, ranges excluding either or both of those included limits are also included in the disclosure. To the extent a discrete value is stated, or an approximation of such value may be claimed, such as “about” said value or “approximately” said value, and this paragraph serves as support for such a claim unless the description explicitly states that such an approximation is not appropriate.

If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

1. A prosthetic valve comprising:
a saddle-shaped annulus;
a pair of support elements connected to the annulus; and
a pair of leaflets connected to the annulus and the support elements,
wherein the annulus and support elements are adapted so that movement of the annulus toward or between different convex and concave states moves ends of the support elements outward and inward relative to each other, and
wherein the leaflets and support elements are adapted to define a valve opening when the support element ends are moved outward and to define a closure when the support element ends are moved inward.

2. The prosthetic valve of claim 1, wherein when the valve is implanted in a mitral position, change between diastole and systole causes the annulus and support element movement resulting in leaflet opening and closure, respectively.

3. The prosthetic valve of claim 1, wherein the valve opening comprises a slot.

4. The prosthetic valve of claim 1, wherein the annulus and the pair of support elements comprise a shape memory material.

5. The prosthetic valve of claim 4, wherein the shape memory material comprises a nickel titanium alloy.

6. The prosthetic valve of claim 1, wherein the leaflets comprise synthetic material selected from engineered biological tissue, biological valvular leaflet tissue, pericardial tissue, cross-linked pericardial tissue, and combinations thereof.

7. The prosthetic valve of claim 6, wherein the leaflets comprise pericardial tissue selected from bovine, equine, porcine, ovine, human tissue, and combinations thereof.

8. The prosthetic valve of claim 6, wherein the leaflets comprise a compliant membrane of a single sheet or a compound manifold.

9. The prosthetic valve of claim 1, wherein the support elements are angled toward one another when the pair of leaflets define the closure, the closure being a seal.

10. The prosthetic valve of claim 1, wherein the support elements are angled away from one another when the pair of leaflets define the opening.

11. A method for prosthetic valve operation, the valve comprising valve leaflets and support arms, wherein the leaflets are connected to the support arms, the method comprising:
opening the valve leaflets by deflecting the support arms outward; and
closing the valve leaflets by deflecting the support arms inward.

12. The method of claim 11, wherein the valve is implanted in a human subject.

13. The method of claim 12, wherein the valve is implanted as a mitral valve replacement adjacent a left ventricle of a heart.

14. The method of claim 13, wherein the valve further comprises an annulus and the method further comprises deflecting the annulus in a direction that is convex toward the left ventricle in diastole to open the valve.

15. The method of claim 14, wherein the valve annulus is in a convex configuration when open.

16. The method of claim 13, wherein the valve further comprises an annulus and the method further comprises deflecting the annulus in a direction that is concave toward the left ventricle in systole to close the valve.

17. The method of claim 16, wherein the valve annulus is in a convex configuration when closed.

18. The method of claim 13, wherein pressure differences in the heart deflect an annulus of the valve for the opening and the closing.
